(19) United States
(12) Patent Application Publication
(10) Pub. No.: US 2018/0334453 A1
(43) Pub. Date: Nov. 22, 2018

(54) ISOINDOLINE DERIVATIVES

(71) Applicant: VIIV HEALTHCARE UK LIMITED, Brentford, Middlesex (GB)

(72) Inventors: Brian Alvin JOHNS, Research Triangle Park, NC (US); Emile Johann VELTHUISEN, Research Triangle Park, NC (US); Jason Gordon WEATHERHEAD, Research Triangle Park, NC (US)

(21) Appl. No.: 15/776,486

(22) PCT Filed: Dec. 1, 2016
(86) PCT No.: PCT/IB2016/057269

§ 371 Date: May 16, 2018

Related U.S. Application Data
(60) Provisional application No. 62/262,938, filed on Dec. 4, 2015.

(51) Int. Cl. C07D 405/04; C07D 209/44; A61P 31/18

(52) U.S. Cl. CPC C07D 405/04; A61P 31/18; C07D 209/44

(57) ABSTRACT

Compounds of Formula I are disclosed and methods of treating viral infections with compositions comprising such compounds.

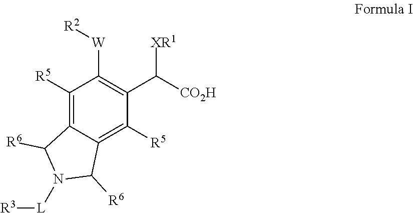

### FIELD OF THE INVENTION

The present invention relates to substituted isoindoline compounds, pharmaceutical compositions, and methods of use thereof for (i) inhibiting HIV replication in a subject infected with HIV, or (ii) treating a subject infected with HIV, by administering such compounds.

### BACKGROUND OF THE INVENTION

Human immunodeficiency virus type 1 (HIV-1) leads to the contraction of acquired immune deficiency disease (AIDS). The number of cases of HIV continues to rise, and currently over twenty-five million individuals worldwide suffer from the virus. Presently, long-term suppression of viral replication with antiretroviral drugs is the only option for treating HIV-1 infection. Indeed, the U.S. Food and Drug Administration has approved twenty-five drugs over six different inhibitor classes, which have been shown to greatly increase patient survival and quality of life. However, additional therapies are still required because of undesirable drug-drug interactions; drug-food interactions; non-adherence to therapy; and drug resistance due to mutation of the enzyme target.

Currently, almost all HIV positive patients are treated with therapeutic regimens of antiretroviral drug combinations termed, highly active antiretroviral therapy (“HAART”). However, HAART therapies are often complex because a combination of different drugs must be administered often daily to the patient to avoid the rapid emergence of drug-resistant HIV-1 variants. Despite the positive impact of HAART on patient survival, drug resistance can still occur. The emergence of multidrug-resistant HIV-1 isolates has serious clinical consequences and must be suppressed with a new drug regimen, known as salvage therapy.

Current guidelines recommend that salvage therapy includes at least two, and preferably three, fully active drugs. Typically, first-line therapies combine three to four drugs targeting the viral enzymes reverse transcriptase and protease. One option for salvage therapy is to administer different combinations of drugs from the same mechanistic class that remain active against the resistant isolates. However, the options for this approach are often limited, as resistant mutations frequently confer broad cross-resistance to different drugs in the same class. Alternative therapeutic strategies have recently become available with the development of fusion, entry, and integrase inhibitors. However, resistance to all three new drug classes has already been reported both in the lab and in patients. Sustained successful treatment of HIV-1-infected patients with antiretroviral drugs will therefore require the continued development of new and improved drugs with new targets and mechanisms of action.

For example, over the last decade HIV inhibitors have been reported to target the protein-protein interaction between HIV-1 integrase and Lens Epithelium Derived Growth Factor/p75 (“LEDGF”). LEDGF is a cellular transcriptional cofactor of HIV-1 integrase that promotes viral integration of reverse transcribed viral cDNA into the host cell's genome by tethering the preintegration complex to the chromatin. Because of its crucial role in the early steps of HIV replication, the interaction between LEDGF and integrase represents another attractive target for HIV drug therapy.

U.S. provisional patent application 62/027,359 discloses certain isoindoline compounds having the following formula:

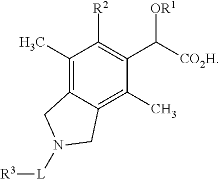

### SUMMARY OF THE INVENTION

Briefly, in one aspect, the present invention discloses compounds of Formula I:

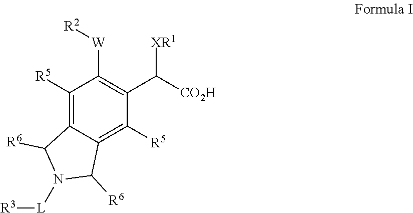

wherein:

X is O or CH₂;

R¹ is C₁₋₆alkyl wherein said alkyl may contain cycloalkyl portions;

W is a bond, —CH═CH—, —C═C—, C₁₋₃alkylene, —CH₂C(O)NH—, —NHC(O)—, —N(CH₃)C(O)—, —N(CH₃)C(O)CH₂—, —C(O)—, —CH₂C(O)—, or —NHC(O)CH₂—, wherein each W is optionally substituted by 1 or 2 methyl groups;

R² is H, C₁₋₆alkyl, C₅₋₁₄aryl, C₃₋₇cycloalkyl, C₃₋₇cycloalkenyl, C₃₋₉heterocycle, or C₅₋₉heteroaryl, wherein each R² group is optionally substituted by one to four substituents selected from halo, C₁₋₆alkyl, C₁₋₆hetereoalkyl, or C₁₋₆alkylene or C₁₋₆hetereoalklylene wherein said C₁₋₆alkylene or C₁₋₆hetereoalklylene is bonded to adjacent carbon atoms on said C₅₋₁₄aryl, C₃₋₇cycloalkyl, C₃₋₇cycloalkenyl, C₃₋₉heterocycle, or C₅₋₉heteroaryl to form a fused ring;

L is a bond, —CH₂(CO)—, —C₁₋₃alkylene—, —SO₂—, —C(O)—, —C(S)—, —C(NH)—, —C(O)NH—, —C(O)NHCH₂—, —C(O)N—, —C(O)OCH₂—, —C(O)O—, —C(O)C(O)—, —SO₂—NH—, or —CH₂C(O)—;

R³ is H, CN, oxo, C₁₋₆alkyl, C₅₋₁₄aryl, CH₂C₅₋₁₄aryl, CH₂C₃₋₇cycloalkyl, C₃₋₇cycloalkyl, C₃₋₇spirocycloalkyl, C₃₋₇cycloalkenyl, C₃₋₉heterocycle, or C₅₋₉heteroaryl, or R³ may join together with an R⁶ to form a fused 5-7 membered ring, and wherein each R³ group is optionally substituted by one to four substituents selected from halo, oxo, C₁₋₆alkyl, C₃₋₇cycloalkyl, C₁₋₃fluoroalkyl, —OC₁₋₆alkyl, —C(O)R⁴, —C(O)NR⁴, —C(O)NHR⁴, C₅₋₁₄aryl, C₁₋₆hetereoalkyl, —B(OH)₂, C₃₋₉heterocycle, C₅₋₉heteroaryl, —C(O)OC₁₋₆alkyl, or two substituents may bond together to form a fused, spiro, or bridged ring and that fused, spiro, or bridged ring may optionally be substituted with R⁴;

R⁴ is CN, halo, —OC₁₋₆alkyl, C₁₋₆alkyl, C₃₋₇cycloalkyl, C₃₋₉heterocycle, or C₅₋₁₄aryl;

each R⁵ is independently H, C₁₋₃alkyl, C₃₋₆cycloalkyl, CH₂F, CHF₂, or CF₃, with the proviso that at least one R⁵ is other than CH₃;

each R⁶ is independently H, or C₁₋₃alkyl, C₅₋₁₄aryl, C₃₋₉heterocycle, C₅₋₉heteroaryl, —C(O)NR⁴, or —C(O)NHR⁴, or both R⁶ may together comprise 2-4 carbon atoms and join together to form a bridged ring system.

and wherein each heterocycle, heteroaryl, heteroalkyl, and heteroalkylene comprises one to three heteroatoms selected from S, N, B, or O.

In another aspect the present invention discloses pharmaceutically acceptable salts of the compounds of Formula I.

In another aspect, the present invention discloses pharmaceutical compositions comprising a compound of Formula I or a pharmaceutically acceptable salt thereof.

In another aspect, the present invention discloses a method for treating a viral infection in a patient mediated at least in part by a virus in the retrovirus family of viruses, comprising administering to said patient a composition comprising a compound of Formula I, or a pharmaceutically acceptable salt thereof. In some embodiments, the viral infection is mediated by the HIV virus.

In another aspect, a particular embodiment of the present invention provides a method of treating a subject infected with HIV comprising administering to the subject a therapeutically effective amount of a compound of Formula I, or a pharmaceutically acceptable salt thereof.

In yet another aspect, a particular embodiment of the present invention provides a method of inhibiting progression of HIV infection in a subject at risk for infection with HIV comprising administering to the subject a therapeutically effective amount of a compound of Formula I, or a pharmaceutically acceptable salt thereof. Those and other embodiments are further described in the text that follows.

In accordance with another embodiment of the present invention, there is provided a method for preventing or treating a viral infection in a mammal mediated at least in part by a virus in the retrovirus family of viruses which method comprises administering to a mammal, that has been diagnosed with said viral infection or is at risk of developing said viral infection, a compound as defined in Formula I, wherein said virus is an HIV virus and further comprising administration of a therapeutically effective amount of one or more agents active against an HIV virus, wherein said agent active against the HIV virus is selected from the group consisting of Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors.

### DETAILED DESCRIPTION OF THE INVENTION

Preferably R¹ is C₁₋₆alkyl. Most preferably, R¹ is t-butyl.

Preferably X is O.

Preferably W is a bond.

Preferably R² is optionally substituted phenyl. Most preferably, R² is phenyl substituted by one to four substituents selected from fluorine, methyl, —CH₂CH₂CH₂O— wherein said —CH₂CH₂CH₂O— is bonded to adjacent carbon atoms on said phenyl to form a bicyclic ring, or —NHCH₂CH₂O— wherein said —NHCH₂CH₂O— is bonded to adjacent carbon atoms on said phenyl to form a bicyclic ring.

Preferably R³ is C₁₋₆alkyl, phenyl, naphthyl, cyclopentyl, cyclohexyl, pyridyl, or tetrahydropyranyl, each of which is optionally substituted by 1-3 substituents selected from halogen, C₁₋₆alkyl, —OC₁₋₆alky, C₁₋₃fluoroalkyl, or phenyl.

Preferably each R⁶ is H.

Preferably the stereochemistry on the carbon to which OR¹ is bound is as depicted below.

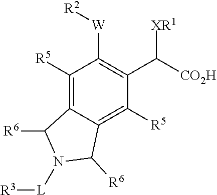

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

### EXAMPLES

The compounds of this invention may be made by a variety of methods, including well-known standard synthetic methods. Illustrative general synthetic methods are set out below and then specific compounds of the invention are prepared in the working examples.

The following examples serve to more fully describe the manner of making and using the above-described invention. It is understood that these examples in no way serve to limit the true scope of the invention, but rather are presented for illustrative purposes. In the examples below and the synthetic schemes above, the following abbreviations have the following meanings. If an abbreviation is not defined, it has its generally accepted meaning.

aq.=aqueous

μL=microliters

μM=micromolar

NMR=nuclear magnetic resonance

boc=tert-butoxycarbonyl

br=broad

Cbz=benzyloxycarbonyl

d=doublet

δ=chemical shift

oC=degrees celcius

DCM=dichloromethane

dd=doublet of doublets

DMEM=Dulbeco's Modified Eagle's Medium

DMF=N,N-dimethylformamide

DMSO=dimethylsulfoxide

EtOAc=ethyl acetate

g=gram

h or hr=hours

HCV=hepatitis C virus

HPLC=high performance liquid chromatography

Hz=hertz

IU=International Units

IC₅₀=inhibitory concentration at 50% inhibition

J=coupling constant (given in Hz unless otherwise indicated)

m=multiplet

M=molar

M+H+=parent mass spectrum peak plus H+

mg=milligram

min=minutes

mL=milliliter

mM=millimolar

mmol=millimole

MS=mass spectrum

nm=nanomolar

ppm=parts per million

q.s.=sufficient amount

s=singlet

RT=room temperature

sat.=saturated

t=triplet

TFA=trifluoroacetic acid

Z=benzyloxycarbonyl

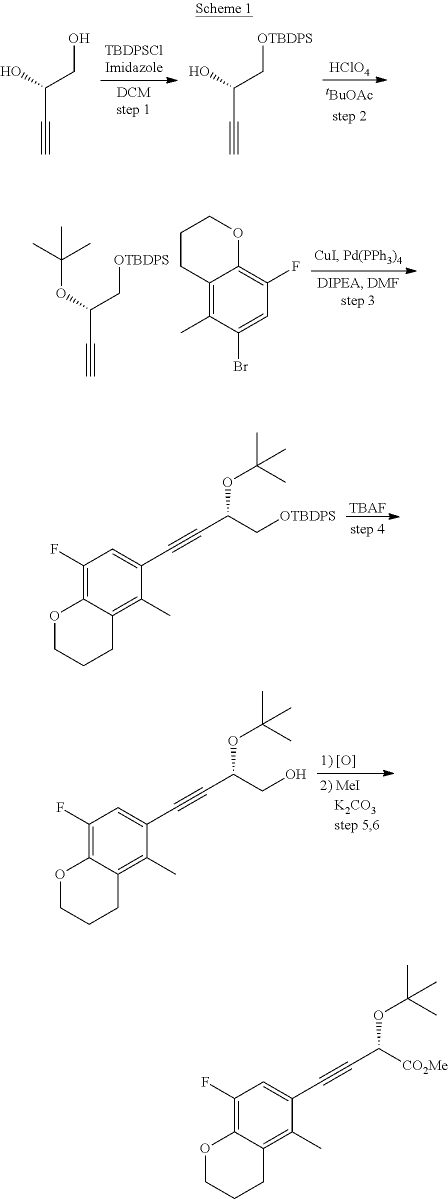

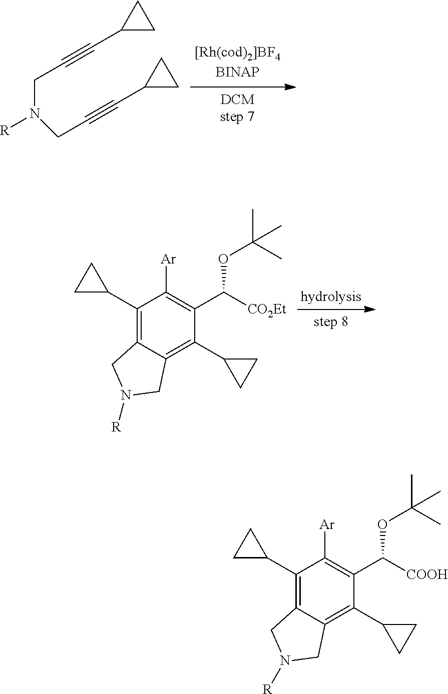

### Example 1

### (2S)(M)-2-ethoxy-2-((R)-6-(8-fluoro-5-methylchroman-6-yl)-2-(3-fluorobenzoyl)-4,7-dimethylisoindolin-5-yl)acetic Acid

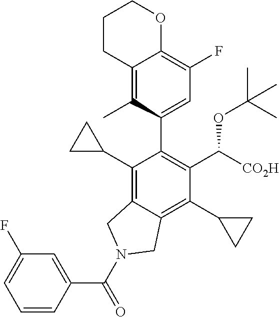

N,N-bis(3-cyclopropylprop-2-yn-1-yl)-3-fluorobenzamide

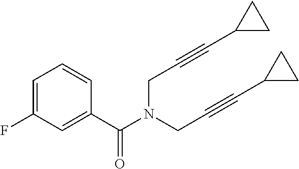 [SMILES: O=C(c1cccc(F)c1)N(CC#CC1CC1)CC#CC1CC1]

To an ice cold solution of 3-fluorobenzamide (100 mg, 0.72 mmol) in DMF (2 mL) was added NaH (72 mg, 1.80 mmol). After 10 min, a solution of 3-cyclopropylprop-2-yn-1-yl methanesulfonate (251 mg, 1.44 mmol) (made according to WO20095674/A2) was added and the reaction mixture warmed to ambient temperature. After 1 h, the reaction mixture was quenched with sat. NH₄Cl aq. and extracted with EtOAc. The organic layer was washed with brine, dried over Na₂SO₄, filtered, and concentrated in vacuo. The residue was purified by silica gel chromatography (0-30% EtOAc in PE) to afford the title compound (44 mg, 21% yield) as a white solid. LC/MS (m/z) ES+=296.1 (M+1).

(S)-But-3-yne-1,2-diol

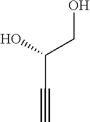

The title compound was prepared from the known procedure as described in WO2010/130034.

6-Bromo-8-fluoro-5-methylchroman

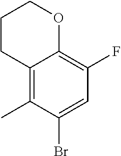

The title compound was prepared from the known procedure as described in WO2010/130842

### Step 1: (S)-1-((tert-Butyldiphenylsilyl)oxy)but-3-yn-2-ol

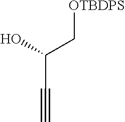

An ice cold solution of (S)-But-3-yne-1,2-diol (220 mg, 2.56 mmol) in DCM (10 mL) was treated with imidazole (209 mg, 3.067 mmol) and TBDPSCI (0.730 mL, 2.812 mmol). After 18 h, the reaction mixture was poured into sat. aq. NaHCO₃ and the layers partitioned. The organic layer was washed with brine, dried (MgSO₄), filtered and concentrated in vacuo. The residue was purified by silica gel chromatography (0-100% EtOAc-hexanes) to afford the title compound (425 mg, 51%) as a colorless oil. 1H NMR (400 MHz, CHLOROFORM-d): δ 1.07 (s, 9 H), 2.41 (d, 1 H), 2.64 (d, 1 H), 3.73 (dd, 1 H), 3.80 (dd, 1 H), 4.45 (m, 1 H), 7.41 (m, 6 H), 7.67 (m, 4 H). LCMS (m/z ES+): 347 (M+23).

Step 2: (S)-((2-(tert-Butoxy)but-3-yn-1-yl)oxy)(tert-butyl)diphenylsilane

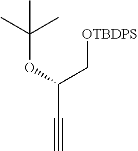

A solution of (S)-1-((tert-Butyldiphenylsilyl)oxy)but-3-yn-2-ol (425 mg, 1.311 mmol) in tert-butyl acetate (70 mL) was treated with HCl₄ (3.93 mL, 1.311 mmol). After 10 min, the reaction mixture was cooled to 0 C and treated with 1 N NaOH until the pH was =7. The reaction mixture was diluted with EtOAc and the layers partitioned. The organic phase was washed with brine, dried (Na₂SO₄), filtered and concentrated in vacuo. The residue was purified by silica gel chromatography (0-100% EtOAc-hexanes) to afford the title compound (470 mg, 95%) as a colorless oil. ¹H NMR (400 MHz, CHLOROFORM-d): δ 1.04 (s, 9 H), 1.24 (s, 9 H), 2.31 (d, 1 H), 3.70 (m, 2 H), 4.24 (m, 1 H), 7.37 (m, 6 H), 7.70 (m, 4 H). LCMS (m/z ES+): 403 (M+23).

Step 3: (S)-((2-(tert-Butoxy)-4-(8-fluoro-5-methylchroman-6-yl)but-3-yn-1-yl)oxy)(tert-butyl)diphenylsilane

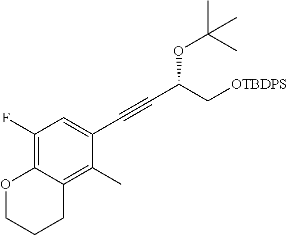

A solution of 6-Bromo-8-fluoro-5-methylchroman (409 mg, 1.68 mmol), (S)-((2-(tert-Butoxy)but-3-yn-1-yl)oxy)(tert-butyl)diphenylsilane (956 mg, 2.516 mmol) and diisopropyl amine (3.59 mL, 252 mmol) in DMF (10 mL) was degassed with N₂ for 10 min and treated with Cul (64 mg, 0.336 mmol) and Pd(PPh₃)₄ (388 mg, 0.336 mmol) and then heated to 80° C. After 18 h, the reaction mixture was diluted with EtOAc. Saturated aqueous NH₄Cl was added and the layers partitioned. The organic phase was washed with water, brine, dried (MgSO₄) filtered and concentrated in vacuo. The residue was purified by silica gel chromatography (0-100% EtOAc-hexanes) to afford the title compound (762 mg, 83%) as a red oil. ¹H NMR (400 MHz, CHLOROFORM-d): δ 1.07 (s, 9 H), 1.29 (s, 9 H), 2.05 (m, 2 H), 2.23 (s, 3 H), 2.63 (t, 2 H), 3.78 (m, 2 H), 4.20 (m, 2 H), 4.51 (dd, 1 H), 6.95 (d, 1 H), 7.39 (m, 6 H), 7.73 (m, 4 H). LCMS (m/z ES+): 567 (M+23).

Step 4: (S)-2-(tert-Butoxy)-4-(8-fluoro-5-methylchroman-6-yl)but-3-yn-1-ol

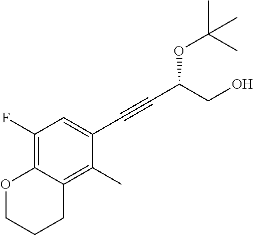 [SMILES: Cc1c(C#C[C@@H](CO)OC(C)(C)C)cc(F)c2c1CCCO2]

A solution of (S)-((2-(tert-Butoxy)-4-(8-fluoro-5-methylchroman-6-yl)but-3-yn-1-yl)oxy)(tert-butyl)diphenylsilane (760 mg, 1.4 mmol) in THF (2 mL) was treated with TBAF (14 mL, 14 mmol, 1.0 M in THF). After 15 min, the reaction mixture was concentrated in vacuo and purified by silica gel chromatography (0-100% EtOAc-hexanes) to afford the title compound (402 mg, 94%) as a colorless oil. ¹H NMR (400 MHz, CHLOROFORM-d): δ 1.34 (s, 9 H), 2.06 (m, 2 H), 2.26 (s, 3 H), 2.65 (t, 2 H), 3.70 (m, 2 H), 4.21 (m, 2 H), 4.48 (dd, 1 H), 6.97 (d, 1 H). LCMS (m/z ES+): 329 (M+23).

Step 5: (S)-2-(tert-Butoxy)-4-(8-fluoro-5-methylchroman-6-yl)but-3-ynoic acid

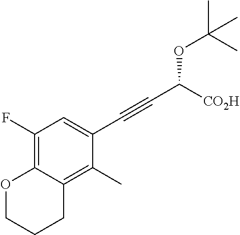 [SMILES: Cc1c(C#C[C@H](OC(C)(C)C)C(=O)O)cc(F)c2c1CCCO2]

A suspension of (S)-((2-(tert-Butoxy)-4-(8-fluoro-5-methylchroman-6-yl)but-3-yn-1-yl)oxy)(tert-butyl)diphenylsilane (108 mg, 0.353 mmol) in DCM (5 mL) was treated with Dess Martin periodinane (300 mg, 0.706 mmol). After 18 h, the reaction mixture was quenched with sat. aq. Na₂S₂O₃ and the layers partitioned. The organic layer was washed with brine, dried (Na₂SO₄), filtered and concentrated in vacuo to afford the title compound as a yellow oil (312 mg) that was used immediately without further purification. LCMS (m/z ES+): 343 (M+23).

Step 6: (S)-methyl 2-(tert-butoxy)-4-(8-fluoro-5-methylchroman-6-yl)but-3-ynoate

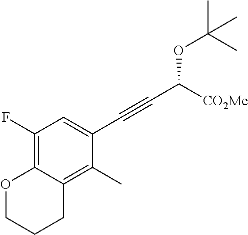 [SMILES: COC(=O)[C@H](C#Cc1cc(F)c2c(c1C)CCCO2)OC(C)(C)C]

A solution of (S)-2-(tert-Butoxy)-4-(8-fluoro-5-methylchroman-6-yl)but-3-ynoic acid (312 mg) and Cs₂CO₃ (171 mg, 0.525 mmol) was treated with Mel (0.110 mL, 1.75 mmol). After 2 h, the reaction mixture was diluted with EtOAc and water. The layers were partitioned and the organic layer was washed with water, brine, dried (MgSO₄), filtered and concentrated in vacuo. The residue was purified by silica gel chromatography (0-100% EtOAc-hexanes) to afford the title compound (40 mg, 32% of 2 steps) as a colorless oil. ¹H NMR (400 MHz, CHLOROFORM-d): δ 1.32 (s, 9 H), 2.06 (m, 2 H), 2.26 (s, 3 H), 2.63 (t, 2 H), 3.83 (s, 3 H), 4.20 (m, 2 H), 4.99 (s, 1 H), 7.00 (d, 1H). LCMS (m/z ES+): 335 (M+1).

Step 7: (2S)(M)-ethyl 2-(tert-butoxy)-2-(-4,7-dicyclopropyl-6-(8-fluoro-5-methylchroman-6-yl)-2-(3-fluorobenzoasoindolin-5-yl)acetate

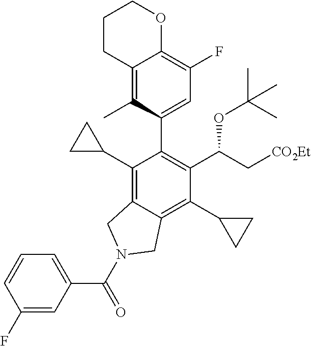

A mixture of R-BINAP (68 mg, 0.11 mmol) and [Rh(cod)₂]BF₄ (45 mg, 0.11 mmol) in DCM (2 mL) was stirred under H₂ atmosphere for 1 hr to generate the activated catalyst. The resulting mixture was purged with N₂ and heated up to 40 ° C. before the addition of methyl (S)-2-(tert-butoxy)-4-(8-fluoro-5-methylchroman-6-yl)but-3-ynoate (120 mg, 0.36 mmol) in DCM (2 mL). A solution of N,N-bis(3-cyclopropylprop-2-yn-1-yl)-3-fluorobenzamide (318 mg, 1.08 mmol) in DCM (6 mL) was added dropwise to the reaction mixture over 30 min and the mixture was stirred at 40° C. for another 30min. The resulting mixture was concentrated under reduced pressure and purified by silica gel chromatography (0-30% EtOAc in PE) to afford the title compound as a yellow oil (40 mg, 18% yield). LCMS (m/z) ES+=630.2 (M+1).

Step 8: (2S)(M)-2-(tert-butoxy)-2-(-4,7-dicyclopropyl-6-(8-fluoro-5-methylchroman-6-yl)-2-(3-fluorobenzoyl)isoindolin-5-yl)acetic acid

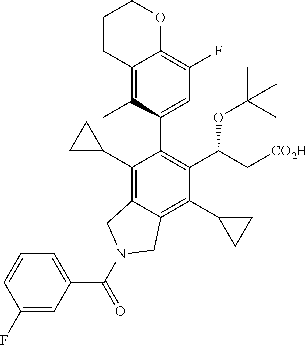

A mixture of methyl (2S)-2-(tert-butoxy)-2-(4,7-dicyclopropyl-6-(8-fluoro-5-methylchroman-6-yl)-2-(3-fluorobenzoyl)isoindolin-5-yl)acetate (40 mg, 0.06 mmol) in dioxane (5 mL) was treated with LiOH (1.27 mL, 1.27 mmol, 1.0 N) and was heated to 80° C. After 18 h, the reaction mixture was cooled to ambient temperature and neutralized with 1N HCl and extracted with DCM/i-PrOH (80:20). The organic layer was washed with brine, dried over Na₂SO₄, filtered and concentrated under reduced pressure. The residue was purified by reverse phase HPLC (C18, 0-100% MeCN in H₂O with 0.1% formic acid) to afford the title compound (4.0 mg, 10% yield) as a white powder. ¹H NMR (400 MHz, DMSO) δ 12.19 (br, 1 H), 7.54 (m, 3 H), 7.36 (dd, J=15.5, 7.5 Hz, 1 H), 6.46 (m, 1 H), 4.85 (m, 5 H), 4.15 (m, 2 H), 2.63 (m, 2 H), 2.00 (m, 2 H), 1.78 (d, J=11.2 Hz, 3 H), 1.36 (m, 2 H), 1.02 (d, J=18.5 Hz, 9 H), 0.53 (m, 8 H). LCMS (m/z) ES+=616.7 (M+1).

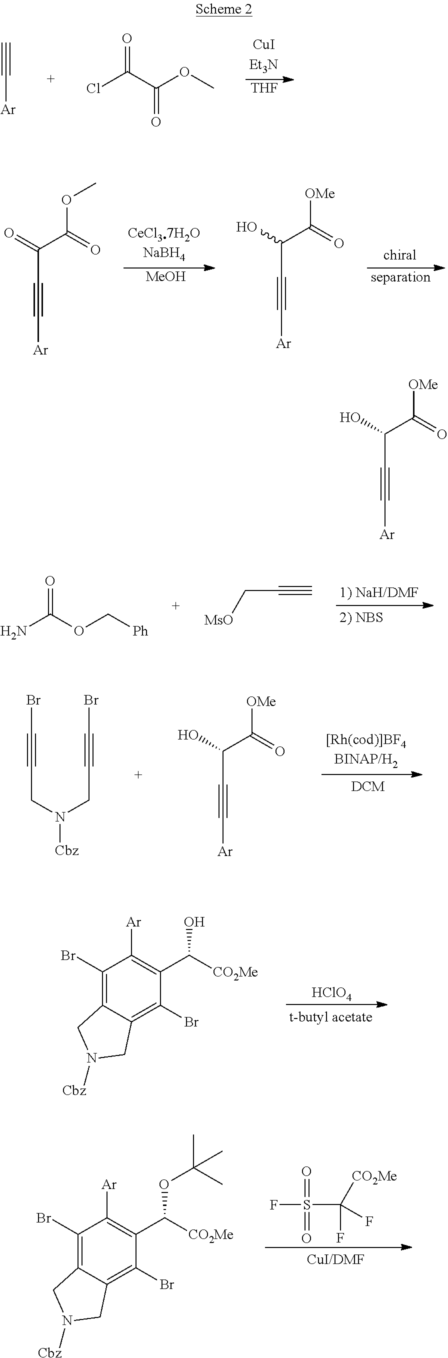

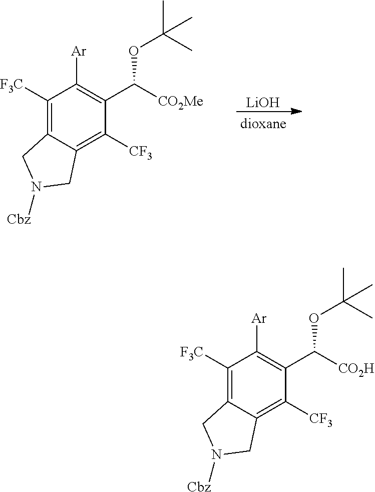

### Example 2

### (S)-2-(2-((benzyloxy)carbonyI)-6-(p-tolyl)-4,7-bis(trifluoromethyl)isoindolin-5-yl)-2-(tert-butoxy)acetic Acid

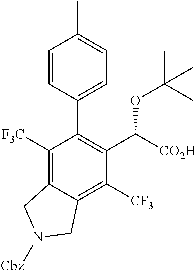 [SMILES: Cc1ccc(-c2c([C@H](OC(C)(C)C)C(=O)O)c(C(F)(F)F)c3c(c2C(F)(F)F)CN(C(=O)OCc2ccccc2)C3)cc1]

Step 1: Methyl 2-oxo-4-(p-tolyl)but-3-ynoate

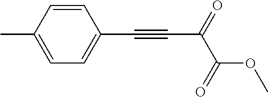

A suspension of Cul (0.1 eq, 1.722 mmol, 0.328 g) in THF (40 mL) was treated with Et₃N (3 eq, 51.7 mmol, 7.20 mL) and stirred until a colorless solution formed. Then, 1-ethynyl-4-methylbenzene (1.0 eq, 17.22 mmol, 2.183 mL) and methyl-2-chloro-2-oxoacetate (2.0 eq, 34.4 mmol, 3.17 mL) were added and the yellow reaction mixture stirred at ambient temperature. After 18 h, the reaction mixture was quenched with sat. aq. NaHCO₃. The aqueous layer was extracted with ethyl acetate (x3). The combined organic layers were washed with brine, dried over Na₂SO₄ and concentrated in vacuo to a brown solid. The crude material was purified via silica gel column chromatography (0-100% EtOAc-hexanes) to afford the title compound as an orange solid (2.32 g, 67% yield). ¹H NMR (400 MHz, CDCl₃) δ 7.58-7.56 (m, 2 H), 7.23-7.21 (m, 2 H), 3.95 (s, 3 H), 2.40 (s, 3 H). LCMS (ES+)(m/z): 203.15 (M+H).

Step 2: (S)-methyl 2-hydroxy-4-(p-tolyl)but-3-ynoate

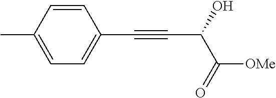

A solution of methyl-2-oxo-4-(p-tolyl)but-3-ynoate (1.0 eq, 200 mg, 0.989 mmol) in methanol (5 mL) was treated with CeCl₃·7 H₂O (1.25 eq, 0.461 g, 1.23 mmol) and then NaBH₄ (0.5 eq, 0.47945 mmol, 19 mg) was added portion wise. After 15 min, the reaction mixture was concentrated in vacuo the residue was quenched with dilute HCl and extracted with DCM (x3). The combined organic layers were washed with brine, dried over Na₂SO₄ and concentrated. The crude material was purified via column chromatography (0-100% EtOAc-hexanes) followed by chiral purification (SFC OD, 5% IPA/CO₂, 140 bar, 40° C., first eluting peak, 4.7 min) to afford the title compound. ¹H NMR (400 MHz, CDCl₃) δ 7.34-7.32 (m, 2 H), 7.12-7.10 (m, 2 H), 5.03 (d, 1H), 4.34 (q, 2 H), 3.07 (d, 1 H), 2.34 (s, 3 H), 1.32 (t, 3 H). LCMS (ES+)(m/z): 219.81 (M+H).

Step 3: benzyl di(prop-2-yn-1-yl)carbamate

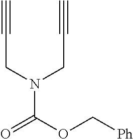 [SMILES: C#CCN(CC#C)C(=O)OCc1ccccc1]

An ice cold suspension of NaH (1.11 g, 27.8 mmol) in DMF (50 mL) was treated with propargyl bromide (3.02 mL, 27.1 mmol, 80 wt % in Toluene) followed by a solution of benzyl carbamate (2.0 g, 13.2 mmol) in DMF (15 mL). After 18 h, the reaction mixture was poured into ice water and extracted with Et2O. The organic layer was washed with water, brine, dried (Na₂SO₄), filtered and concentrated in vacuo. The residue was purified by silica gel chromatography (0-40% EtOAc/hexanes) to afford the title compound (1.75 g, 58%) as a yellow oil. ¹H NMR (400 MHz, CHLOROFORM-d) δ ppm 7.43-7.29 (m, 5 H), 5.19 (s, 2 H), 4.27 (br. s., 4 H), 2.26 (t, J=2.5 Hz, 2 H); LCMS (m/z) ES+=228 (M+1).

Step 4: benzyl di(prop-2-yn-1-yl)carbamate

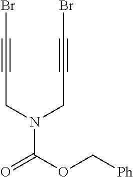 [SMILES: O=C(OCc1ccccc1)N(CC#CBr)CC#CBr]

An ice cold solution of benzyl di(prop-2-yn-1-yl)carbamate (535 mg, 2.354 mmol) in acetone (12 mL) was shielded from light and treated with NBS (838 mg, 4.71 mmol) and silver nitrate (160 mg, 0.942 mmol). After 100 min, the reaction mixture was diluted with EtOAc and washed with sat. aq. Na₂S₂O₃ and sat. aq. NaHCO₃. The layers were partitioned and the organic layer washed with brine, dried (Na₂SO₄), filtered and concentrated in vacuo to afford the title compound as a yellow oil that was used without further purification. LCMS (m/z) ES+=381.8 (M−1).

Step 5: (S)-benzyl 4,7-dibromo-5-(2-methoxy-1-hydroxy-2-oxoethyl)-6-(p-tolyl)isoindoline-2-carboxylate

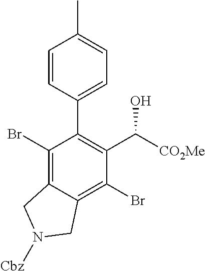 [SMILES: COC(=O)[C@@H](O)c1c(Br)c2c(c(Br)c1-c1ccc(C)cc1)CN(C(=O)OCc1ccccc1)C2]

A two-necked round bottom flask was charged with [Rh(cod)2]BF₄ (0.084 g, 0.206 mmol) and (+/−)-BINAP (0.128 g, 0.206 mmol) in anhydrous DCM (4 mL) was sparged with H₂ for 5 minutes and stirred under 1 atm (balloon) of H₂. After 1 hour the reaction mixture was sparged with N₂ and treated with a solution of (S)-methyl 2-hydroxy-4-(p-tolyl)but-3-ynoate (210 mg, 1.028 mmol) in dichloromethane (1 mL) and placed in a preheated 50° C. oil bath. The reaction mixture was then treated dropwise with a solution of benzyl bis(3-bromoprop-2-yn-1-yl)carbamate (594 mg, 1.542 mmol) in dichloromethane (3 mL) over 85 min. After 30 min, the reaction mixture was cooled to ambient temperature and concentrated in vacuo and purified by silica gel chromatography (0-60% EtOAc-hexanes) to afford the title compound (0.52 g, 85%) as a yellow oil. LCMS (m/z) ES+=610 (M+23).

Step 6: (S)-benzyl 4,7-dibromo-5-(1-(tert-butoxy)-2-methoxy-2-oxoethyl)-6-(p-tolyl)isoindoline-2-carboxylate

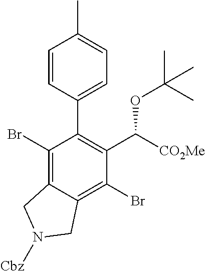 [SMILES: COC(=O)[C@@H](OC(C)(C)C)c1c(Br)c2c(c(Br)c1-c1ccc(C)cc1)CN(C(=O)OCc1ccccc1)C2]

A solution of (S)-benzyl 4,7-dibromo-5-(1-hydroxy-2-methoxy-2-oxoethyl)-6-(p-tolyl)isoindoline-2-carboxylate (516 mg, 0.876 mmol) in tert-butyl acetate (9 mL, 66.6 mmol) was treated dropwise with perchloric acid (0.301 mL, 3.504 mmol). After 15 min, the mixture was quenched with aq. 2 M NaOH and sat. NaHCO3, extracted with EtOAc, washed with brine, dried over Na₂SO₄, filtered, and concentrated in vacuo. The residue was purified by silica gel chromatography (0-50% EtOAc/Hexane) to afford the title compound (157.2 mg, 0.244 mmol, 25.6% yield) as colorless oil. 1H NMR (400 MHz, CHLOROFORM-d) δ ppm 7.48-7.30 (m, 5 H), 7.24 (t, J=7.1 Hz, 2 H), 7.16 (d, J=7.6 Hz, 1 H), 7.10-6.98 (m, 1 H), 5.30-5.12 (m, 3 H), 4.97-4.74 (m, 4 H), 3.68 (s, 3 H), 2.43 (s, 3 H), 1.01 (d, J=3.6 Hz, 9 H); LCMS (m/z) ES+=666 (M+23).

Step 7: (S)-benzyl 5-(1-(tert-butoxy)-2-methoxy-2-oxoethyl)-6-(p-tolyl)-4,7-bis(trifluoromethyl)isoindoline-2-carboxylate

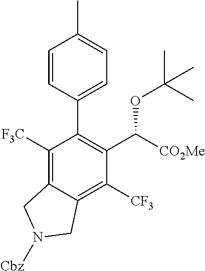 [SMILES: COC(=O)[C@@H](OC(C)(C)C)c1c(-c2ccc(C)cc2)c(C(F)(F)F)c2c(c1C(F)(F)F)CN(C(=O)OCc1ccccc1)C2]

A solution of methyl 2,2-difluoro-2-(fluorosulfonyl)acetate (268 mg, 1.395 mmol) and (S)-benzyl 4,7-dibromo-5-(1-(tert-butoxy)-2-methoxy-2-oxoethyl)-6-(p-tolyl)isoindoline-2-carboxylate (90 mg, 0.139 mmol) in N,N-Dimethylformamide (DMF) (3 mL) was treated with copper(I) iodide (106 mg, 0.558 mmol) and warmed to 115° C. After 2 h, the reaction mixture was treated with additional methyl 2,2-difluoro-2-(fluorosulfonyl)acetate (170 mg) and Cul (50 mg). After 1.5 h, the reaction mixture was cooled to ambient temperature, filtered through acrodisc ptfe filter, and washed with EtOAc. The filtrate was washed with water, brine, dried over Na₂SO₄, filtered, and concentrated in vacuo. The residue was purified by silica gel chromatography (0-40% EtOAc/Hexane) to afford the title compounds (71.3 mg, 82% yield) as light pink oil. ¹H NMR (400 MHz, CHLOROFORM-d) δ ppm 7.49-7.31 (m, 5 H), 7.25 (d, J=8.4 Hz, 1 H), 7.21-7.08 (m, 3 H), 5.33-5.20 (m, 2 H), 5.13-4.86 (m, 5 H), 3.69 (s, 3 H), 2.43 (s, 3 H), 0.95 (s, 9 H); LCMS (m/z) ES⁺=646.49 (M+Na).

Step 8: (S)-2-(2-((benzyloxy)carbonyl)-6-(p-tolyl)-4,7-bisarifluoromethyl)isoindolin-5-yl)-2-(tert-butoxy)acetic acid.

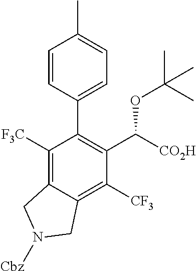 [SMILES: Cc1ccc(-c2c([C@H](OC(C)(C)C)C(=O)O)c(C(F)(F)F)c3c(c2C(F)(F)F)CN(C(=O)OCc2ccccc2)C3)cc1]

A solution of (S)-benzyl 5-(1-(tert-butoxy)-2-methoxy-2-oxoethyl)-6-(p-tolyl)-4,7-bis(trifluoromethyl)isoindoline-2-carboxylate (7.0 mg, 0.011 mmol) in 1,4-dioxane (0.5 mL) was treated with LiOH (0.056 mL, 0.112 mmol, 2.0 M) and stirred at 70° C. After 24 h, the reaction mixture was treated with additional 2 M LiOH (0.056 mL, 0.112 mmol, 2.0 M) and stirred at 70 ° C. After 18 h, the reaction mixture was cooled to ambient temperature and concentrated in vacuo. The residue was purified by reverse phase HPLC (20-100% MeCN/H2O-0.1% TFA) to afford the title compound (2 mg, 3.12 pmol, 27.8% yield) as white solid. ¹H NMR (400 MHz, METHANOL-d4) δ ppm 7.53-7.15 (m, 9 H), 5.27 (s, 2 H), 5.08-4.94 (m, 6 H), 2.46 (s, 3 H), 0.96 (s, 9 H); LCMS (m/z) ES⁺=632.43 (M+23).

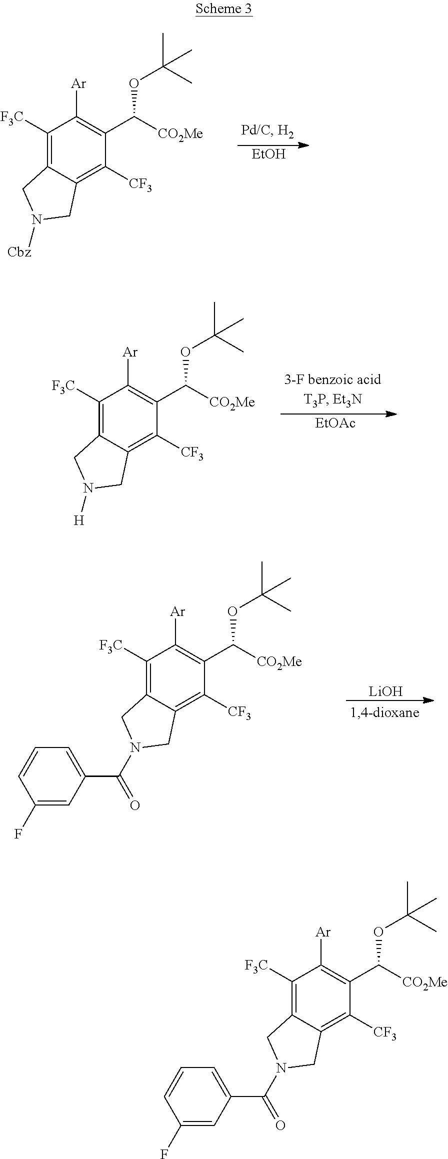

### Example 3. (S)-2-(tert-butoxy)-2-(2-(3-fluorobenzoyl)-6-(p-tolyl)-4,7-bisarifluoromethyl)isoindolin-5-yl)acetic Acid

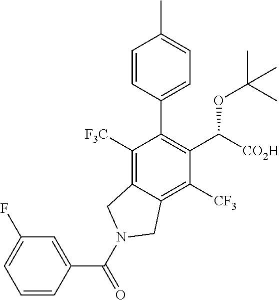 [SMILES: Cc1ccc(-c2c([C@H](OC(C)(C)C)C(=O)O)c(C(F)(F)F)c3c(c2C(F)(F)F)CN(C(=O)c2cccc(F)c2)C3)cc1]

Step 1: (S)-methyl 2-(tert-butoxy)-2-(6-(p-tolyl)-4,7-bisarifluoromethyl)isoindolin-5-yl)acetate

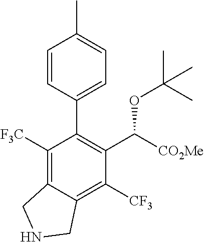 [SMILES: COC(=O)[C@@H](OC(C)(C)C)c1c(-c2ccc(C)cc2)c(C(F)(F)F)c2c(c1C(F)(F)F)CNC2]

A solution of (S)-benzyl 5-(1-(tert-butoxy)-2-methoxy-2-oxoethyl)-6-(p-tolyl)-4,7-bis(trifluoromethyl)isoindoline-2-carboxylate (56 mg, 0.090 mmol) in ethanol (2 mL) was purged and filled with N₂, treated with Pd/C (10 wt %, degussa) (9.56 mg, 8.98 pmol), and then bubbled with H₂ for 3 min and placed under a balloon of H₂ (1 atm). After 50 min, the reaction mixture was filtered through a pad of celite, washed with MeOH, EtOH and DCM, and then concentrated in vacuo to afford the title compound (54 mg) that was used without further purification. LCMS (m/z) ES+=490.4 (M+H).

Step 2: (S)-methyl 2-(tert-butoxy)-2-(2-(3-fluorobenzoyl)-6-(p-tolyl)-4,7-bisarifluoromethyl)isoindolin-5-yl)acetate

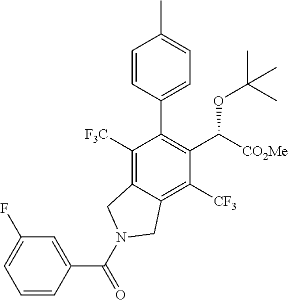 [SMILES: COC(=O)[C@@H](OC(C)(C)C)c1c(-c2ccc(C)cc2)c(C(F)(F)F)c2c(c1C(F)(F)F)CN(C(=O)c1cccc(F)c1)C2]

A solution of (S)-methyl 2-(tert-butoxy)-2-(6-(p-tolyl)-4,7- bis(trifluoromethyl)isoindolin-5-yl)acetate (50 mg, 0.102 mmol) in ethyl acetate (2.5 mL) was treated with 3-fluorobenzoic acid (21.47 mg, 0.153 mmol), Et₃N (0.043 mL, 0.306 mmol), propane phosphonic acid anhyrdide (0.152 mL, 0.255 mmol, 50 wt % in EtOAc). After 1 h, the reaction was diluted with sat. NaHCO₃, extracted with EtOAc, washed with bBrine, dried over Na₂SO₄, filtered, and concentrated in vacuo to give afford the title compound (60.8 mg, 0.099 mmol, 97% yield) as brown oil. LCMS (m/z) ES+=612.49 (M+1).

Step 3: (S)-2-(tert-butoxy)-2-(2-(3-fluorobenzoyl)-6-(p-tolyl)-4,7-bis(trifluoromethyl)isoindolin-5-yl)acetic acid

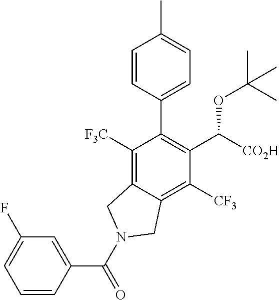 [SMILES: Cc1ccc(-c2c([C@H](OC(C)(C)C)C(=O)O)c(C(F)(F)F)c3c(c2C(F)(F)F)CN(C(=O)c2cccc(F)c2)C3)cc1]

A solution of (S)-methyl 2-(tert-butoxy)-2-(2-(3-fluorobenzoyl)-6-(p-tolyl)-4,7-bis(trifluoromethyl)isoindolin-5-yl)acetate (60.8 mg, 0.099 mmol, 97% yield) in 1,4-Dioxane (2.5 mL) was treated with LiOH (0.511 mL, 1.022 mmol, 2.0 M) and stirred at 70° C. After 18 h, the mixture was cooled ambient temperature and concentrated in vacuo. The residue was purified by reverse phase HPLC (15-95% MeCN/H₂O-0.1% TFA) to afford a mixture of products that was redissolved in 1,4-dioxane (0.75 mL) and ethanol (0.75 mL), treated with 2 M LiOH (0.612 mL, 1.224 mmol), and stirred at 85 ° C. After 72 h, the reaction was cooled to ambient temperature and concentrated in vacuo to afford (S)-2-(tert-butoxy)-2-(6-(p-tolyl)-4,7-bis(trifluoromethyl)isoindolin-5-yl)acetic acid, LCMS (m/z) ES+=476.42 (M+1).

The residue was suspended in ethyl acetate (1.5 mL), treated with 3-fluorobenzoic acid (12.88 mg, 0.092 mmol), Et₃N (0.026 mL, 0.183 mmol), propane phosphonic acid anhyrdide (0.091 mL, 0.153 mmol, 50wt % in EtOAc), and stirred at ambient temperature. After 80 min, the reaction mixture was diluted with 1 N HCl, extracted with EtOAc, washed with brine, dried over Na₂SO₄, filtered, and concentrated in vacuo. The residue was purified by reverse phase HPLC (15-85% MeCN/H2O-0.1% TFA) to afford the title compound (10.8 mg, 0.018 mmol, 17.16% yield) as an off-white solid. NMR showed rotomers. ¹H NMR (400 MHz, CHLOROFORM-d) δ ppm 7.55-7.43 (m, 2 H), 7.39 (d, J=7.5 Hz, 1 H), 7.32 (d, J=8.8 Hz, 1 H), 7.29-7.18 (m, 3 H), 7.15-7.02 (m, 1 H), 5.56-5.36 (m, 1 H), 5.22-4.80 (m, 4 H), 2.43 (d, J=3.6 Hz, 3 H), 0.99 (d, J=5.9 Hz, 9 H); LCMS (m/z) ES+=598.48 (M+1).

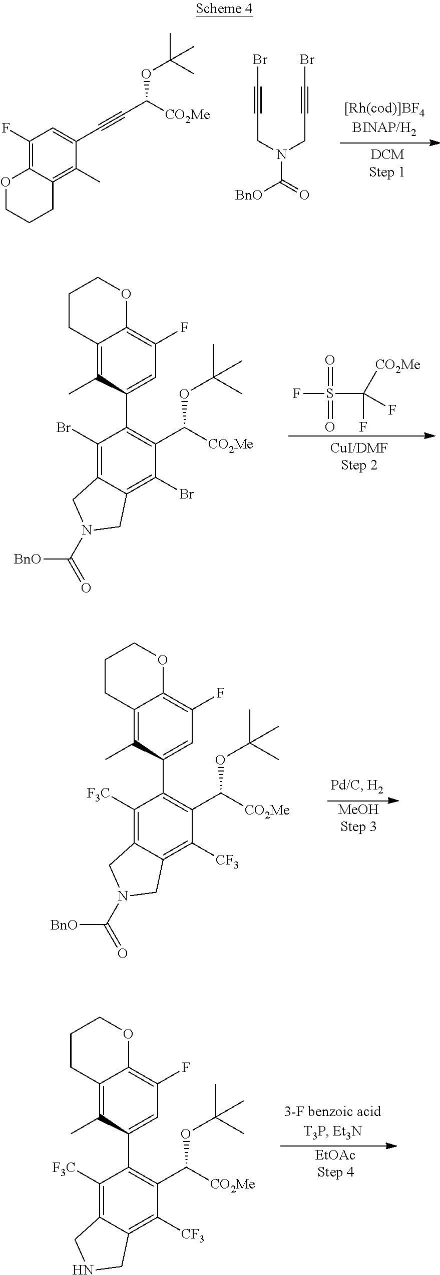

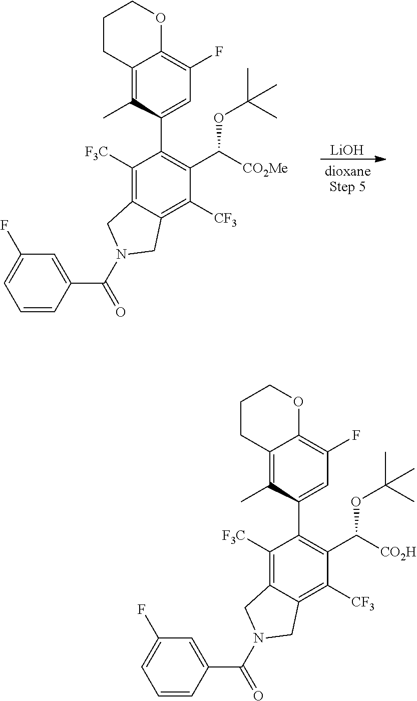

### Example 4

### (2S)(M)-2-(tert-butoxy)-2-(6-(8-fluoro-5-methylchroman-6-yl)-2-(3-fluorobenzoyl)-4,7-bisarifluoromethasoindolin-5-yl)acetic Acid

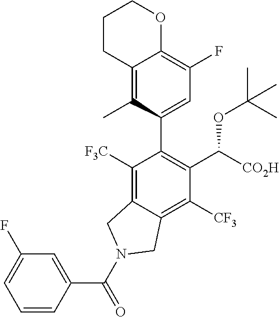

Step 1: (S)-benzyl 4,7-dibromo-5-((S)-1-(tert-butoxy)-2-methoxy-2-oxoethyl)-6-(8-fluoro-5-methylchroman-6-asoindoline-2-carboxylate

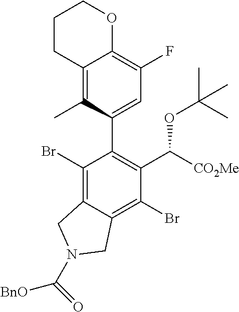

A two-neck round bottom flask was charged with [Rh(cod)₂]BF₄ (72.9 mg, 0.179 mmol) and (R)-BINAP (112 mg, 0.179 mmol). The mixture was dissolved with dichloromethane (DCM) (3 mL), degassed with H₂ for 5 min, and then stirred under an atmosphere of H2. After 1 h, the reaction mixture was flushed with N₂ and treated with a solution of (S)-methyl 2-(tert-butoxy)-4-(8-fluoro-5-methylchroman-6-yl)but-3-ynoate (300 mg, 0.897 mmol) in dichloromethane (DCM) (1.5 mL). A condenser was added and the mixture was placed in a preheated 50° C. bath, and treated dropwise with a solution of benzyl bis(3-bromoprop-2-yn-1-yl)carbamate (518 mg, 1.346 mmol) in dichloromethane (2.5 mL) over 80 min. The mixture was refluxed for 1.5 hours, and then cooled to ambient temperature. After 18 h, an additional 0.09 mmol of the prepared catalyst was added to the reaction, followed by a solution of benzyl bis(3-bromoprop-2-yn-1-yl)carbamate (336 mg) in DCM (1.5 mL) dropwise over 1.5 hours. The mixture was refluxed for 1 h, cooled to ambient temperature, and then concentrated in vacuo. The residue was purified by silica gel chromatography (0-50% EtOAc/Hexane) to afford the title compound (176.2 mg, 0.245 mmol, 27.3% yield) as light yellow oil. ¹H NMR (400 MHz, CHLOROFORM-d) δ ppm 7.48-7.30 (m, 5 H), 6.46-6.34 (m, 1 H), 5.44 (d, J=10.9 Hz, 1 H), 5.24 (d, J=3.6 Hz, 2 H), 4.97-4.78 (m, 4 H), 4.27 (t, J=5.0 Hz, 2 H), 3.64 (s, 3 H), 2.74-2.61 (m, 2 H), 2.16-2.07 (m, 2 H), 1.85 (s, 3 H), 1.10 (s, 9 H); LCMS (m/z) ES+=742.36 (M+23).

Step 2: (2S)(M)-benzyl 5-(-1-(tert-butoxy)-2-methoxy-2-oxoethyl)-6-(8-fluoro-5-methylchroman-6-yl)-4,7-bisarifluoromethasoindoline-2-carboxylate

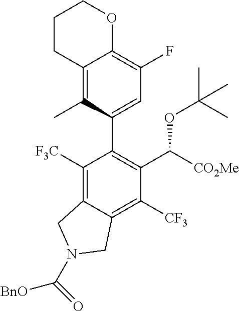

A solution of methyl 2,2-difluoro-2-(fluorosulfonyl)acetate (454 mg, 2.363 mmol) and benzyl 4,7-dibromo-5-((S)-1-(tert-butoxy)-2-methoxy-2-oxoethyl)-6-(8-fluoro-5-methylchroman-6-yl)isoindoline-2-carboxylate (170 mg, 0.236 mmol) in N,N-dimethylformamide (DMF) (5 mL) was treated with copper(I) iodide (180 mg, 0.945 mmol) and stirred at 115° C. After 2 h, the reaction mixture was treated with additional methyl 2,2-difluoro-2-(fluorosulfonyl)acetate (454 mg, 2.363 mmol), copper(I) iodide (180 mg, 0.945 mmol), and stirred at 115° C. After 1 h, additional methyl 2,2-difluoro-2-(fluorosulfonyl)acetate (454 mg, 2.363 mmol), copper(I) iodide (180 mg, 0.945 mmol), was added and stirring continued at 115° C. After 1 h, the reaction mixture was cooled to ambient temperature, filtered, and washed with EtOAc. The filtrate was washed with water, brine, dried over Na₂SO₄, filtered, and concentrated in vacuo. The residue was purified by silica gel chromatography (0-100% EtOAc/Hexane) to afford benzyl 5-((S)-1-(tert-butoxy) -2-methoxy-2-oxoethyl)-6-(8-fluoro-5-methylchroman-6-yl)-4,7-bis(trifluoromethyl)isoindoline-2-carboxylate (107.6 mg, 0.154 mmol, 65.3% yield) as light yellow oil. LCMS (m/z) ES+=720.55 (M+Na).

Step 3: (2S)(M)-methyl 2-(tert-butoxy)-2-(-6-(8-fluoro-5-methylchroman-6-yl)-4,7-bisarifluoromethasoindolin-5-yl)acetate

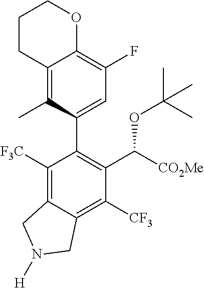

A solution (2S)(M)-benzyl 5+1-(tert-butoxy)-2-methoxy-2-oxoethyl)-6-(8-fluoro-5-methylchroman-6-yl)-4,7-bis(trifluoromethyl)isoindoline-2-carboxylate (105 mg, 0.151 mmol) in methanol (2 mL) was purged and filled with N₂, treated with Pd/C (10 wt %, degussa) (16.0 mg, 0.015 mmol), and then bubbled with H₂ for 3 min. The reaction was stirred at ambient temperature and under an atmosphere of H₂. After 1.5 h, the mixture was diluted with ethanol (1 mL), Pd/C (10 wt %, degussa) (16.02 mg, 0.015 mmol), bubbled with H₂ for 2 min, and then stirred at ambient temperature and under an atmosphere of H₂. After 2.5 h, the mixture was flushed with N₂ and filtered through a pad of celite, washed with MeOH, EtOAc and DCM, and then concentrated in vacuo to give crude the title compound (73.6 mg, 0.131 mmol, 87% yield) as dark yellow oil. The crude product was used as is. LCMS (m/z) ES+=564.43 (M+1).

Step 4: (S)-methyl 2-(tert-butoxy)-2-((S)-6-(8-fluoro-5-methylchroman-6-yl)-2-(3-fluorobenzoyl)-4,7-bis(trifluoromethyl)isoindolin-5-yl)acetate

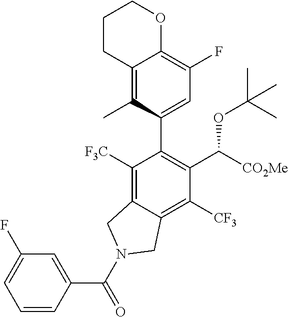

A solution of crude (2S)(M)-methyl 2-(tert-butoxy)-2-(6-(8-fluoro-5-methylchroman-6-yl)-4,7-bis(trifluoromethyl)isoindolin-5-yl)acetate (73.6 mg, 0.131 mmol, 87% yield) in ethyl acetate (3 mL) was treated with 3-fluorobenzoic acid (31.6 mg, 0.226 mmol), Et₃N (0.063 mL, 0.452 mmol), propane phosphonic acid anhyrdide (0.224 mL, 0.376 mmol, 50 wt % in EtOAc), and stirred at ambient temperature. After 72 h, the reaction was diluted with sat. NaHCO₃, extracted with EtOAc, washed with Brine, dried over Na₂SO₄, filtered, and concentrated in vacuo. The residue was purified by silica gel chromatography (0-70% EtOAc/Hexane) to afford the title compound (37 mg, 0.054 mmol, 35.9% yield) as pinkish red oil. NMR showed rotomers. ¹H NMR (400 MHz, CHLOROFORM-d) δ ppm 7.56-7.43 (m, 1 H), 7.38 (d, J=7.3 Hz, 1 H), 7.31 (d, J=9.3 Hz, 1 H), 7.25-7.16 (m, 1 H), 6.69 (br. s., 1 H), 5.41-4.90 (m, 5 H), 4.37-4.19 (m, 2 H), 3.71-3.52 (m, 3 H), 2.76-2.53 (m, 2 H), 2.20-2.08 (m, 2 H), 1.74 (br. s., 3 H), 1.13-0.97 (m, 9 H); LCMS (m/z) ES+=686.45 (M+1).

Step 5: (S)-2-(tert-butoxy)-2-((S)-6-(8-fluoro-5-methylchroman-6-yl)-2-(3-fluorobenzoyl)-4,7-bis(trifluoromethyl)isoindolin-5-yl)acetic acid

A solution of (2S)(M)-methyl 2-(tert-butoxy)-2-(6-(8-fluoro-5-methylchroman-6-yl)-2-(3-fluorobenzoyl)-4,7-bis(trifluoromethyl)isoindolin-5-yl)acetate (37 mg, 0.054 mmol) in 1,4-dioxane (1.1 mL) was treated with KOTMS (27.7 mg, 0.216 mmol) and stirred at 100° C. After 1 h, the reaction mixture was treated with additional KOTMS (27.7 mg, 0.216 mmol) and stirred at 100° C. for 5.5 h, and then cooled to ambient temperature over 18 h. The reaction mixture was partitioned between EtOAc and 1 N HCl and the organic layer washed with brine, dried over Na₂SO₄, filtered, and concentrated in vacuo. The residue was dissolved in tetrahydrofuran (0.75 mL) and methanol (0.75 mL), treated with LiOH (0.540 mL, 1.08 mmol, 2.0 M), and stirred at 85° C. After 2.5 h, the reaction was cooled to ambient temperature and concentrated in vacuo to afford crude (2S)(M)-2-(tert-butoxy)-2-(6-(8-fluoro-5-methylchroman-6-yl)-4,7-bis(trifluoromethyl)isoindolin-5-yl)acetic acid [LCMS (m/z) ES+=550.42 (M+1)]. The residue was suspended in EtOAc (1.5 mL), treated with 3-fluorobenzoic acid (11.35 mg, 0.081 mmol), Et3N (0.023 mL, 0.162 mmol), propane phosphonic acid anhyrdide (0.080 mL, 0.135 mmol, 50wt % in EtOAc), and stirred at ambient temperature. After 1.5 h, the reaction mixture was diluted with 1 N HCl, extracted with EtOAc, washed with brine, dried over Na₂SO₄, filtered, and concentrated in vacuo. The residue was purified by reverse phase HPLC (25-90% MeCN/H₂O-0.1% TFA) to afford the title compound (4.6 mg, 6.16 pmol, 11.42% yield) as light brown solid. ¹H NMR (400 MHz, CHLOROFORM-d) δ ppm 7.55-7.44 (m, 1 H), 7.39 (d, J=7.7 Hz, 1 H), 7.32 (d, J=8.7 Hz, 1 H), 7.26-7.18 (m, 1 H), 6.78 (br. s., 1 H), 5.48-5.33 (m, 1 H), 5.28-5.13 (m, 2 H), 5.12-4.86 (m, 2 H), 4.35-4.21 (m, 2 H), 2.75-2.57 (m, 2 H), 2.18-2.07 (m, 2 H), 1.85 (s, 3 H), 1.17-1.05 (m, 9 H); LCMS (m/z) ES+=672.49 (M+1).

Anti-HIV Activity

MT4 Assay

Antiviral HIV activity and cytotoxicity values for compounds of the invention from Table 1 were measured in parallel in the HTLV-1 transformed cell line MT-4 based on the method previously described (Hazen et al., 2007, In vitro antiviral activity of the novel, tyrosyl-based human immunodeficiency virus (HIV) type 1 protease inhibitor brecanavir (GW640385) in combination with other antiretrovirals and against a panel of protease inhibitor-resistant HIV (Hazen et al., “In vitro antiviral activity of the novel, tyrosyl-based human immunodeficiency virus (HIV) type 1 protease inhibitor brecanavir (GW640385) in combination with other antiretrovirals and against a panel of protease inhibitor-resistant HIV”, Antimicrob. Agents Chemother. 2007, 51: 3147-3154; and Pauwels et al., “Sensitive and rapid assay on MT-4 cells for the detection of antiviral compounds against the AIDS virus”, J. of Virological Methods 1987, 16: 171-185).

Luciferase activity was measured 96 hours later by adding a cell titer glo (Promega, Madison, Wis.). Percent inhibition of cell protection data was plotted relative to no compound control. Under the same condition, cytotoxicity of the compounds was determined using cell titer Glo™ (Promega, Madison, Wis.). IC₅₀ s were determined from a 10 point dose response curve using 3-4-fold serial dilution for each compound, which spans a concentration range >1000 fold.

These values are plotted against the molar compound concentrations using the standard four parameter logistic equation:

y=((Vmax*x̂n)/(K̂n+x̂n))+Y2

where:

Y2=minimum y n=slope factor

Vmax=maximum y x=compound concentration [M]

K=EC₅₀

When tested in the MT4 assay compounds were found to have IC₅₀ values listed in Table 1.

TABLE 1

| Example | IC₅₀ (uM) |
|---|---|
| 1 | 0.145 |
| 2 | 4.40 |
| 3 | 0.005 |
| 4 | 0.004 |

1. A compound of Formula I or a pharmaceutically acceptable salt thereof: 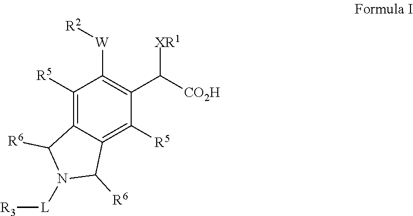
wherein:
X is O or CH₂;
R¹ is C₁₋₆alkyl wherein said alkyl may contain cycloalkyl portions;
W is a bond, —CH═CH—, C₁₋₃alkylene, —CH₂C(O)NH—, —NHC(O)—, —N(CH₃)C(O)—, —N(CH₃)C(O)CH₂—, —C(O)—, —CH₂C(O)—, or —NHC(O)CH₂—, wherein each W is optionally substituted by 1 or 2 methyl groups;
R² is H, C₁₋₆alkyl, C₅₋₁₄aryl, C₃₋₇cycloalkyl, C₃₋₇cycloalkenyl, C₃₋₉heterocycle, or C₅₋₉heteroaryl, wherein each R² group is optionally substituted by one to four substituents selected from halo, C₁₋₆alkyl, C₁₋₆hetereoalkyl, or C₁₋₆alkylene or C₁₋₆hetereoalklylene wherein said C₁₋₆alkylene or C₁₋₆hetereoalklylene is bonded to adjacent carbon atoms on said C₅₋₁₄aryl, C₃₋₇cycloalkyl, C₃₋₇cycloalkenyl, C₃₋₉heterocycle, or C₅₋₉heteroaryl to form a fused ring;
L is a bond, —CH₂(CO)—, —C₁₋₃alkylene—, —SO₂—, —C(O)—, —C(S)—, —C(NH)—, —C(o)NH—, —C(O)NHCH₂—, —C(O)N—, —C(O)OCH₂—, —C(O)O—, —C(O)C(O)—, —SO₂—NH—, or —CH₂C(O)—;
R³ is H, CN, C₁₋₆alkyl, C₅₋₁₄aryl, CH₂C₅₋₁₄aryl, CH₂C₃₋₇cycloalkyl, C₃₋₇cycloalkyl, C₃₋₇spirocycloalkyl, C₃₋₇cycloalkenyl, C₃₋₉heterocycle, or C₅₋₉heteroaryl, oxo, or R³ may join together with R⁶ or R⁷ to form a fused 5-7 membered ring, and wherein each R³ group is optionally substituted by one to four substituents selected from halo, oxo, C₁₋₆alkyl, C₃₋₇cycloalkyl, C₁₋₃fluoroalkyl, —OC₁₋₆alkyl, —C(O)R⁴, —C(O)NR⁴, —C(O)NHR⁴, C₅₋₁₄aryl, C₁₋₆hetereoalkyl, —B(OH)₂, C₃₋₉heterocycle, C₅₋₉heteroaryl, —C(O)OC₁₋₆alkyl, or two substituents may bond together to form a fused, spiro, or bridged ring and that fused, spiro, or bridged ring may optionally be substituted with R⁴;
R⁴ is CN, halo, —OC₁₋₆alkyl, C₁₋₆alkyl, C₃₋₇cycloalkyl, C₃₋₉heterocycle, or C₅₋₁₄aryl;
each R⁵ is independently H, C₁₋₃alkyl, C₃₋₆cycloalkyl, CH₂F, CHF₂, or CF₃, with the proviso that at least one R⁵ is other than CH₃;
each R⁶ is independently H, or C₁₋₃alkyl, C₅₋₁₄aryl, C₃₋₉heterocycle, C₅₋₉heteroaryl, —C(O)NR⁴, or —C(O)NHR⁴, or both R⁶ may together comprise 2-4 carbon atoms and join together to form a bridged ring system.
and wherein each heterocycle, heteroaryl, heteroalkyl, and heteroalkylene comprises one to three heteroatoms selected from S, N, B, or O.

2. A compound or salt according to claim 1 wherein R¹ is C₁₋₆alkyl.

3. A compound or salt according to claim 1 wherein X is O.

4. A compound or salt according to claim 1 wherein W is a bond.

5. A compound or salt according to claim 1 wherein R² is optionally substituted phenyl.

6. A compound or salt according to claim 5 wherein R² is phenyl substituted by one to four substituents selected from fluorine, methyl, —CH₂CH₂CH₂O— wherein said —CH₂CH₂CH₂O— is bonded to adjacent carbon atoms on said phenyl to form a bicyclic ring, or —NHCH₂CH₂O— wherein said —NHCH₂CH₂O— is bonded to adjacent carbon atoms on said phenyl to form a bicyclic ring.

7. A compound or salt according to claim 1 wherein R³ is C₁₋₆alkyl, phenyl, naphthyl, cyclopentyl, cyclohexyl, pyridyl, or tetrahydropyranyl, each of which is optionally substituted by 1-3 substituents selected from halogen, C₁₋₆alkyl, —OC₁₋₆alkyl, C₁₋₃fluoroalkyl, or phenyl.

8. A compound or salt according to claim 1 wherein each R⁶ is H.

9. A compound or salt according to claim 1 wherein the stereochemistry on the carbon to which XR¹ is bound is as depicted below.

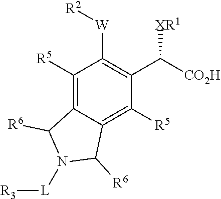

10. (canceled)

11. A pharmaceutical composition comprising a compound or salt according to claim 1.

12. A method for treating a viral infection in a patient mediated at least in part by a virus in the retrovirus family of viruses, comprising administering to said patient a composition according to claim 11.

13. The method of claim 12 wherein said viral infection is mediated by the HIV virus.

14-16. (canceled)
